(19) United States
(12) Patent Application Publication
(10) Pub. No.: US 2011/0290008 A1
(43) Pub. Date: Dec. 1, 2011

(54) NOVEL TECHNIQUE FOR UNIFORMLY APPLYING ANALYTE TO A STRUCTURED SURFACE

(76) Inventors: Steven M. Ebstein, Newton, MA (US); Howard Guthermann, Newton, MA (US)

(21) Appl. No.: 13/149,363

(22) Filed: May 31, 2011

Related U.S. Application Data
(60) Provisional application No. 61/349,499, filed on May 28, 2010.

(51) Int. Cl. G01N 33/00

(52) U.S. Cl. 73/61.43; 252/408.1

(57) ABSTRACT

Nanostructured sensing substrates (nanodevices) offer greatly enhanced sensitivity and selectivity for detection of molecular species through a variety of sensing modalities. In order to produce repeatable and quantifiable assays, it is desirable to apply the analyte uniformly to the nanodevice. Uniform analyte application is promoted by applying the analyte in a fluid mixture or solution which uniformly wets the nanostructured device. The fluid, or mixture of fluids, is chosen to both wet the nanodevice and dissolve or uniformly suspend the analyte.

### REFERENCE TO PENDING PRIOR PATENT APPLICATION

This patent application claims benefit of pending prior U.S. Provisional Patent Application Ser. No. 61/349,499, filed May 28, 2010 by Steven M. Ebstein et al. for NOVEL TECHNIQUE FOR UNIFORMLY APPLYING ANALYTE TO A STRUCTURED SURFACE (Attorney's Docket No. EBSTEIN-4 PROV), which patent application is hereby incorporated herein by reference.

### FEDERALLY SPONSORED RESEARCH

This invention was made with U.S. Government support under Contract W911NF-09-C-0055 awarded by the U.S. Army Research Office. The U.S. Government has certain rights in the invention.

### FIELD OF THE INVENTION

The present invention pertains to a novel technique for applying analytes to substrates for quantitatively detecting and distinguishing between molecules, especially biomolecules, with high sensitivity and selectivity.

### BACKGROUND OF THE INVENTION

Molecular diagnostics are an increasingly important part of chemical sensing and biotechnology. The ability to detect small quantities of chemicals, genomic material, peptides and proteins, metabolites, and other biological materials enables sensitive clinical tests to be performed, as well as enabling laboratory research that affects drug development and functional biology. Molecular sensing modalities include radioactivity, mass spectroscopy, and electrical and optical techniques. The worldwide market for molecular biomarkers, diagnostics, and related services exceeded $9 billion in 2007.

While current diagnostic technology is quite useful, it is not as sensitive or as specific as is desired, and there is room for improvement in the speed, economics, and information content of the diagnostics. Consequently, there is great interest in techniques that improve the ability to unambiguously detect specific molecules in very small quantities.

Nanotechnology has been identified as one approach that can potentially improve the sensitivity of molecular diagnostics.

A variety of approaches using nanotechnology are being developed for molecular sensing which incorporate finely structured surfaces. These include surface enhanced modalities such as Surface Enhanced Raman Spectroscopy (SERS), which relies on excitation of plasmon modes of metal nanoparticles to enhance various optical scattering interactions, such as is described in U.S. Pat. No. 7,586,601 (Attorney's Docket No. EBSTEIN-1), which patent is hereby incorporated herein by reference. There are also other detection methods that rely on an unenhanced but significantly large change in properties when a small amount of material, the analyte, interacts with a nanometer-sized feature of the sensor. For example, the optical properties of a porous silicon device change when a small amount of biomolecules adsorb onto, or bond to, a target molecule fixed to the device. In this and related instances, a change can occur in the refractive index of the surface or the optical path distance (OPD), which can be sensed by evanescent wave measurements or surface plasmon resonance (SPR) measurements.

For many such molecular sensing assays, the analyte must be applied to the finely structured surface in order for the diagnostic procedure to be performed. Inasmuch as many such methods incorporate nanorough or nanostructured surfaces, for the sake of convenience, we shall sometimes hereinafter refer to the apparatus containing such a structured surface as a nanodevice (however, it should also be appreciated that it is not intended that the present invention be limited to nanodevices, since the present invention is also applicable to devices with structured surfaces on the micrometer scale, etc.). In most instances, the analyte is in a fluid or liquid that is applied to the nanodevice. In some instances, the analyte remains in the fluid, i.e., in solution. In other instances, the analyte adsorbs onto the nanodevice and the fluid may or may not be present when the sensing takes place, i.e., the fluid may evaporate or be otherwise removed from the nanodevice.

It is well known that surfaces with significant roughness or structure on micrometer and nanometer scales have different wetting properties than smoother surfaces formed out of the same materials. In particular, the ability to wet a surface generally decreases with increasing roughness on micron and nanometer scales, with some material properties related to wetting varying with increasing roughness. This can affect the ability of a fluid to wet a device with such a surface, and hence can affect the uniformity with which the analyte is applied to such a surface. This phenomenon can be significant in diagnostic applications, where it can be important to uniformly apply the analyte to a structured surface in order to provide superior diagnostic results. It is also well known that surface chemistry can affect wetting properties and that chemical treatments can be applied that make a surface attract or repel a fluid.

One property related to wetting is the contact angle of a drop of fluid on the surface. See FIG. 1, which shows how a drop of fluid placed on a surface forms a contact angle with that surface in general, a contact angle of 90 degrees or less (or some other threshold) indicates favorable wetting, i.e., where a drop will cover a large area of the surface. This threshold for the contact angle is sometimes used to discriminate whether a fluid wets a surface. The contact angle typically increases with increasing surface roughness. Large contact angles generally indicate poor wetting of the surface. Such angles are associated with drops that do not uniformly spread out across the surface and hence result in non-uniform application of an analyte to a structured surface.

In order to produce repeatable and quantifiable assays, it is generally desirable to apply the analyte uniformly to the nanodevice. Techniques which increase the ability to apply an analyte uniformly to the nanodevice are the subject of this invention.

### SUMMARY OF THE INVENTION

In accordance with the present invention, a nanodevice is provided with a region intended to receive the analyte, the region having a finely structured surface.

In one form of the invention, a fluid is selected for its ability to wet the nanodevice. The analyte is mixed into the fluid to form a solution or uniform suspension. The resulting mixture is then applied to the nanodevice. By selecting a fluid with enhanced wetting characteristics, the analyte can be more uniformly dispersed across the nanodevice, thereby enabling improved diagnostic results to be achieved.

In another form of the invention, a composite fluid comprises a mixture of at least two miscible fluids. The function of one fluid is to wet the nanodevice, and the function of a second fluid is to dissolve or uniformly suspend the analyte. The composite fluid, consisting of a mixture of the miscible fluids and the analyte, wets the nanodevice and places the analyte (in solution or suspension) in contact with the nanodevice, thereby enabling more uniform disposition of the analyte on the nanodevice, and hence enabling improved diagnostic results to be achieved.

In one preferred form of the present invention, there is provided a method for applying an analyte to a structured surface, wherein the method comprises:

selecting a fluid that wets the structured surface;

mixing the analyte into the fluid;

applying the mixture to the structured surface.

In another preferred form of the present invention, there is provided a method for performing a diagnostic assay of an analyte, wherein the method comprises:

selecting a fluid that wets a structured surface;

mixing the analyte into the fluid;

applying the mixture to the structured surface; and

performing a diagnostic assay on the analyte.

In another preferred form of the present invention, there is provided a composition for use in performing a diagnostic assay of an analyte, wherein the composition comprises:

a fluid that wets a structured surface, the analyte being mixed into the fluid.

### BRIEF DESCRIPTION OF THE DRAWINGS

These and other objects, features and advantages of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing how a drop of fluid deposited on a surface forms a contact angle θ with a flat surface and a nanostructured surface;

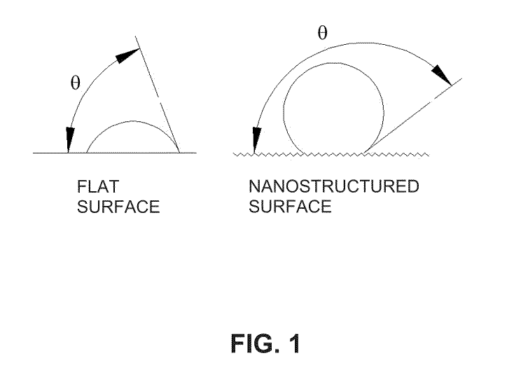

FIG. 2 is an image of an exemplary nanodevice comprising a ˜60 nm Ag coating on an array of silicon nanopillars viewed at 45 degree inclination with 100 nm white scale bar;

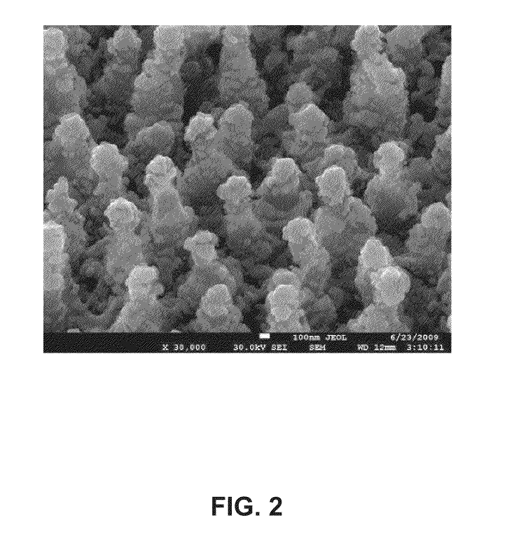

FIG. 3 is a schematic view of a solution being applied to a nanodevice by a pipette;

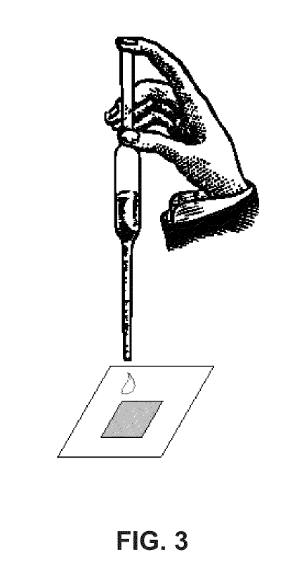

FIG. 4 is an image of a structured surface comprising a 75 nm Ag coating on nanostructured silicon;

FIG. 5 is an image of a structured surface comprising nanostructured silicon;

FIG. 6 is an image of a structured surface comprising nanostructured stainless steel;

FIG. 7 is an image of a structured surface comprising nanostructured copper;

FIG. 8 is an image of a structured surface comprising nanostructured silicon; and

FIG. 9 is an image of a structured surface comprising nanostructured silicon.

### DETAILED DESCRIPTION OF THE PRESENT INVENTION

Nanodevices may be formed in a variety of ways.

Many materials, e.g., silicon, can be patterned by subtractive processes such as photolithographic techniques which selectively etch away portions of the material after a photosensitive material, such as photoresist, has been exposed with a patterned illumination. Materials can be roughened by chemical etching. Materials can be etched with ion beams, either after patterning the surface to selectively etch different regions, or by using a masked or magnetically steered beam.

Materials can also be patterned with an additive process whereby molecules are applied to the surface by a variety of techniques such as evaporative coating, with structures formed either by pre-treating the surface or by self-assembling processes.

Materials can also be structured by laser processing including laser etching (a subtractive process), laser ablation (also a subtractive process), and surface modification techniques whereby a layer of material is melted and re-solidified. The laser processing can produce a pattern by selectively irradiating portions of the original surface, either by translating a fine spot relative to the surface, or by using a fine mask to selectively pass the beam, or by irradiating a large area and relying on random irregularities and/or instabilities to produce the structure.

Laser processing can be performed with lasers having different wavelengths, continuous wave or pulsed beams of different durations, and a range of spot sizes, beam power, and pulse energies. The laser beam can irradiate the surface or substrate material in air; or in. vacuum; or in a chamber where a gaseous fluid is in contact with the material, with the constituent gas or gases chosen, along with their pressures, to impart certain properties to the structured surface. Alternatively, the laser beam can irradiate the material with an intervening layer of a liquid consisting of one or more solvents, e.g., water, alcohol, or oil, and possibly a dopant in the liquid which deposits on, or reacts with, the structured surface.

For many applications, the nanodevice is further processed after a structured surface has been created. For example, the nanodevice can be cleaned with solvents or a vapor or a gaseous plasma. The nanodevice can have a surface layer transformed by reaction with a fluid such as an acid etch, a reactive vapor, or a gaseous plasma. The processing can also involve one or more materials being deposited on the surface. These materials can be dielectric coatings or metals whose composition, thickness, and order are selected so as to modify the properties of the surface after such coatings are applied. These coatings may be applied with any of a number of standard techniques including thermal or electron beam evaporation or sputtering in a vacuum apparatus, electrodeposition, chemical deposition, or other chemical or physical vapor deposition processes including atomic layer deposition. The deposited materials can also be polymer materials that are applied in a variety of ways including dip coating, spin coating, vacuum thin film deposition, or as Langmuir-Blodgett films.

For the purposes of the present invention, the specific manner in which the structured surface of the nanodevice is created is generally not material—what is important is that the nanodevice comprises a structured surface.

After appropriate preparation of the nanodevice, including the preparation of the finely structured surface which is to receive an analyte, the analyte is applied to the nanodevice, typically for the purpose of measuring some property such as identity, composition, amount, or concentration. It is typically desirable to have the analyte uniformly adsorb or otherwise deposit on the nanostructured device in order to provide enhanced diagnostic results. The ability of the analyte to do so may depend on the properties of the surface and the analyte, especially if it is entrained in a fluid as a solution or suspension.

It is well known that micro-structured and nano-structured surfaces can have different wetting properties than fiat surfaces of the same materials. In fact, some liquids, e.g., water, bead up more readily on a structured surface, due to a larger contact angle, than when the surface is flat.

Moreover, it is observed that discontinuities in the surface properties can often confine a solution. For example, a nanostructured region of an otherwise flat material which has a drop of a solvent or solution spotted on it may repel the drop so that the drop stays off the nanostructure or may confine the drop such that the drop cannot cross the boundaries of the nanostructured region.

Discontinuities in the surface properties can also be produced by discontinuities of the surface material. Patterning the surface surrounding a structured region with a different material is one way to accomplish this. If the material outside the structured region has less affinity for the solvent, the drop will be confined to the structured region. For example, a hydrophobic material such as a fluoropolymer surrounding a structured region will tend to confine an aqueous drop to the structured region. Forming an isolated, structured region surrounded by a different material can be accomplished either with an additive process that adds a second material or with a subtractive process that starts with a layered structure formed of two materials and removes some of the outer layer, thereby leaving a structured region surrounded by a second material.

It is generally desirable to apply analytes uniformly to surfaces such as the nanostructured surfaces used for surface enhanced Raman spectroscopy (SERS) in order to provide enhanced diagnostic results. Such nanostructured surfaces and their use in SERS applications are described. in U.S. Pat. No 7,586,601 (Attorney's Docket No. EBSTEIN-1), U.S. patent application Ser. No. 12/584,574 (Attorney's Docket No. EBSTEIN-1 CON) and U.S. patent application Ser. No. 12/430,599 (Attorney's Docket No EBSTEIN-2), which patent and patent applications are hereby incorporated herein by reference. The uniform application of the analyte to the nanostructured surface removes a variable (i.e., the density or uniformity of the applied analyte on the structured surface) from the problem of quantifying the detection response as a function of the analyte areal density on the structured surface which in turn reflects the concentration or amount of the analyte. Thus, the uniform application of the analyte to the structured surface results in enhanced diagnostic results.

The present invention recognizes that the uniform application of an analyte to a structured surface is promoted by the use of an analyte solution or analyte suspension that properly wets the surface and is confined by means other than the surface tension and wetting characteristics of a drop on a homogeneous surface. Such enhanced wetting leads to a more uniform deposition of the solute on the structured surface than the classic scenario where a drop is spotted onto a surface, is held in place by surface tension, and which forms a “coffee ring stain” when the solvent evaporates. As a result, proper wetting of the structured surface with the analyte solution or analyte suspension can result in more uniform distribution of the analyte on the structured surface, which can in turn result in improved diagnostic results.

For a given surface, wetting properties vary for different fluids. For instance, we have found that the laser-nanostructured surfaces discussed in U.S. Pat. No. 7,586,601 and U.S. patent applications Ser. Nos. 12/584,574 and 12/430,599 in connection with their use for SERS applications are not properly wet by water—the drop stays beaded up—but are wet by many organic solvents such as methanol.

Thus, in one form of the invention, a fluid is selected for its ability to wet the structured surface of the nanodevice. The analyte is mixed into the fluid so as to form a solution or uniform suspension. The resulting mixture is then applied to the nanodevice. By selecting a fluid with enhanced wetting characteristics, the analyte can be more uniformly dispersed across the nanodevice, thereby enabling improved diagnostic results to be achieved.

In one preferred form of the invention, the fluid is a solvent.

In one preferred form of the invention, the solvent is an organic solvent.

In one preferred form of the invention, the organic solvent is methanol.

In another form of the invention, a first fluid is selected for its ability to wet the nanodevice, and a second fluid is selected for its ability to dissolve or uniformly suspend the analyte, with the first and second fluids also being miscible with one another. The analyte is mixed into the second fluid so as to form a solution or uniform suspension, and the solution uniform suspension is mixed with the first fluid so as to form a composite fluid. The mixture of the two fluids and the analyte wets the nanodevice and places the analyte (in solution or in uniform suspension) in contact with the nanodevice, thereby enabling more uniform disposition of the analyte on the nanodevice, and hence-enabling improved diagnostic results to be achieved.

Significantly, solvents with different wetting properties, e.g., water and alcohols, are miscible.

In one preferred form of the invention, the first and second fluids are both solvents.

In one preferred form of the invention, one of the first and second fluids is methanol.

Thus, the present invention recognizes that a solvent mixture can be used for uniformly wetting a nanostructured substrate and uniformly applying the analyte, which is in solution, to the surface.

Thus it will be seen that, with the present invention, an analyte can be applied to the nanostructured surface by selecting a solvent that dissolves the analyte and simultaneously wets the structured surface; or (b) providing a composite fluid which comprises at least two miscible fluids, wherein the function of one fluid is to properly wet the device and the function of the second liquid is to dissolve (or suspend) the analyte.

In one preferred form of the invention, there is provided an aqueous solution of the analyte and adding methanol in sufficient ratio so that the resulting aqueous-methanol solution wets the nanostructure; and spotting the solution onto the nanostructure which will be uniformly wet so the analyte has the opportunity to adsorb to the entire surface.

This novel approach of selecting a solvent with appropriate wetting properties, or mixing solvents so the result both wets the structured surface and dissolves the analyte, has been employed by us to uniformly apply analyte to nanostructured surfaces such as those used for SERS.

It should be apparent that the solvents, the ratio of solvents in the case of a mixture, and other means to confine the spotted solution to the nanostructured region are selected based on the properties of the surface, the solvents, the analytes, as well as the design of the process whereby the analyte is presented to the surface. Furthermore, the solvents are selected so as to be compatible with the instrumentation such that the appropriate amounts of solution can be accurately metered when it is applied to the nanodevice.

### EXAMPLES

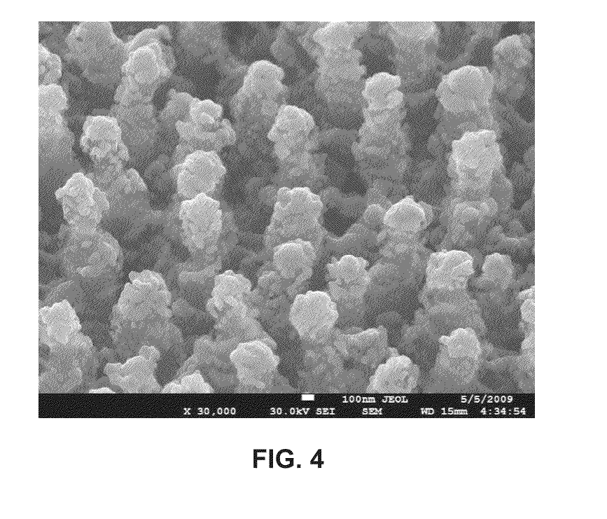

Table 1 shows, for a variety of structured surfaces, the approximate threshold, ratio that allows a structured surface to become substantially completely covered (wet) by a water/methanol solution. FIGS. 4-9 are images of various ones of the structured surfaces listed in Table 1.

### MODIFICATIONS

It will be understood that many additional changes in the details, materials, steps and arrangements of parts, which have been herein described and illustrated in order to explain the nature of the invention, may be made by those skilled in the art while remaining within the principles and scope of the present invention.
This table shows the approximate threshold ratio that allows the Nano Structure to become completely covered (wet) by a Water/Methanol solution. Note that structures labeled with Non-NS were tested as a control surface.

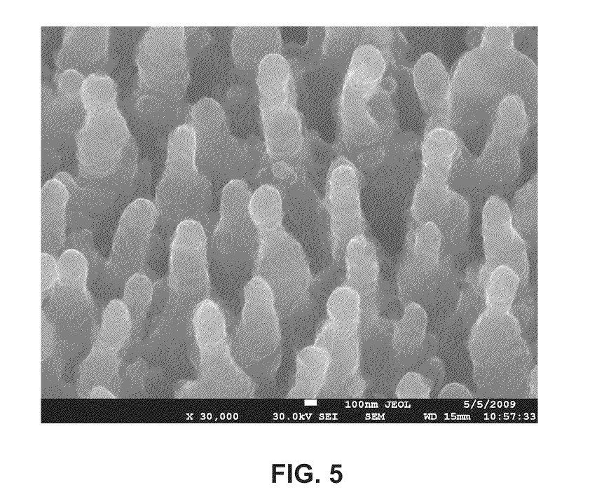

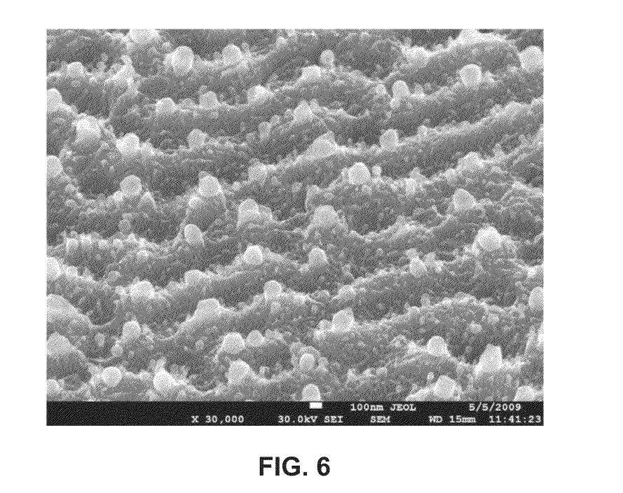

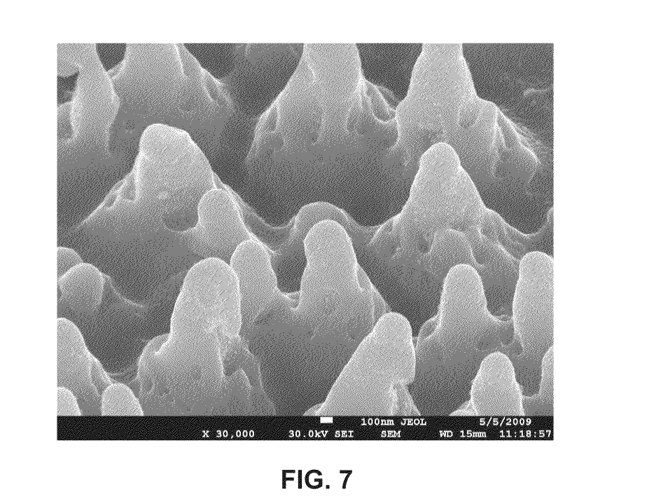

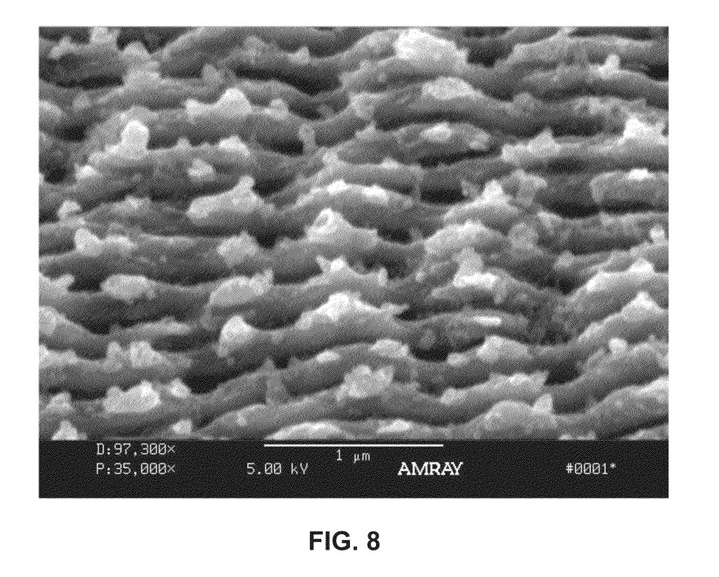

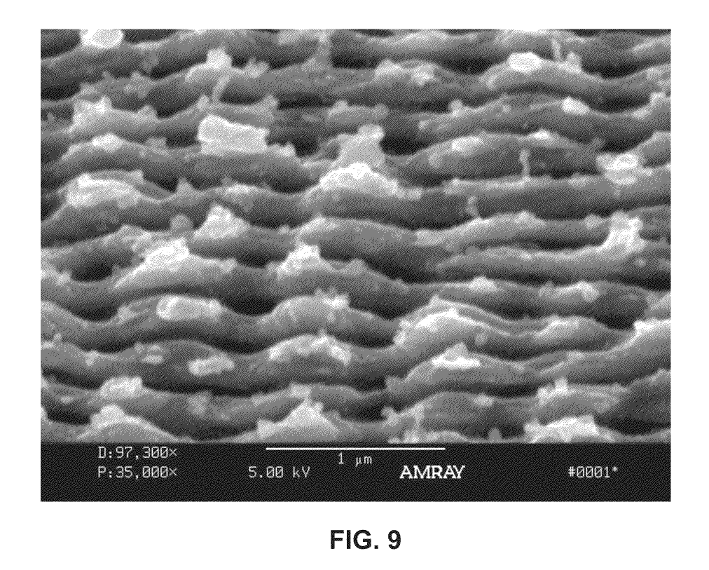

TABLE 1
Ratio of Water/Methanol Solution

| Structure | 90/10 | 75/25 | 70/30 | 50/50 | 25/75 | 10/90 | 0/100 | Feature | width | height | spacing | FIG. No. |
|---|---|---|---|---|---|---|---|---|---|---|---|---|
| Si-05-UR (Silvered NS) | — | — | Bead | Wet | — | — | — | pillar | 372 | 974 | 688 | FIG. 4 |
| Si (Non-NS) | — | — | — | Bead | — | — | — | | | | | |
| Si-05-LR (Nonsilvered NS) | Large | — | Wet | Wet | — | — | — | pillar | 275 | 1392 | 694 | FIG. 5 |
| | Bead* | | | | | | | | | | | |
| Si (Non-NS) | — | — | — | Bead | — | — | — | | | | | |
| SS-03-UR (NS) | — | Bead | — | Bead | Bead | Wet | Wet | bump | 166 | 239 | 503 | FIG. 6 |
| Silvered SS (Non-NS) | — | — | — | Bead | — | — | Bead | | | | | |
| SS (Non-NS) | — | — | — | Bead | — | — | Large | | | | | |
| | | | | | | | Bead* | | | | | |
| Cu-03-UR (NS) | — | Bead | — | Bead | Bead | Wet | Wet | rounded | 544 | 1171 | 928 | FIG. 7 |
| | | | | | | | | cone | | | | |
| Silvered Cu (Non-NS) | — | — | — | Bead | — | — | Bead | | | | | |
| Cu (Non-NS) | — | — | — | Bead | — | — | Bead | | | | | |
| 2 Si-1L | — | Bead | — | Bead | Large | Wet | Wet | bump/ridge | 421/253 | 245 | 788/253 | FIG. 8 |
| | | | | | Bead* | | | | | | | |
| 2 Si-1B | — | Bead | — | Bead | Wet | Wet | Wet | bump/ridge | 421/253 | 245 | 788/253 | FIG. 9 |

NS-Nanostructured Section
Non-NS-Section of the structure that does not contain a Nanostructure (used as a control)
Si-Silicon
SS-Stainless Steel
Cu-Copper
*A large bead means the solution almost wet the structure but did not wet the structure completely
Units are nm
Silver coating is 75 nm applied by thermal evaporation
Cu and SS have a threshold that is between 75%-90% Methanol
Si-05-UR has a threshold that is between 50%-70% Water
Si-05-LR has a threshold that is between 70%-90% Water
2 Si-1L has a threshold that is between 10%-25% Water
2 Si-1B has a threshold tha is between 25%-50% Water

What is claimed is:

1. A method for applying an analyte to a structured surface, wherein the method comprises:
selecting a fluid that wets the structured surface;
mixing the analyte into the fluid;
applying the mixture to the structured surface.

2. A method according to claim 1 wherein the fluid is a solvent for the analyte.

3. A method according to claim 2 wherein the solvent is an organic solvent.

4. A method according to claim 3 wherein the organic solvent is methanol.

5. A method according to claim 1 wherein the analyte is uniformly suspended in the fluid.

6. A method according to claim 1 wherein the fluid comprises a mixture of two or more fluids, at least one of which wets the structured surface.

7. A method according to claim 6 wherein at least one fluid wets the structured surface and at least one fluid mixes with the analyte to form a solution.

8. A method according to claim 7 wherein at least one fluid is a solvent for the analyte, and further wherein the solvent is an organic solvent.

9. A method according to claim 8 wherein the organic solvent is methanol.

10. A method according to claim 7 wherein the analyte is uniformly suspended in at least one fluid.

11. A method according to claim 1 where the structured surface comprises a region of a first material which is surrounded by a region of a second material so as to confine the mixture to the structured region.

12. A method according to claim 1 wherein the fluid comprises water and methanol.

13. A method according to claim 12 wherein the fluid comprises 10% water and 90% methanol.

14. A method for performing a diagnostic assay of an analyte, wherein the method comprises:
selecting a fluid that wets a structured surface;
mixing the analyte into the fluid;
applying the mixture to the structured surface; and
performing a diagnostic assay on the analyte.

15. A method according to claim 14 wherein the fluid is a solvent for the analyte.

16. A method according to claim 14 wherein the analyte is uniformly suspended in the fluid.

17. A method according to claim 14 wherein the fluid comprises a mixture of two or more fluids, at least one of which wets the structured surface.

18. A method according to claim 17 wherein at least one fluid wets the structured surface and at least one fluid mixes with the analyte to form a solution.

19. A method according to claim 17 wherein at least one fluid wets the structured surface and at least one fluid mixes with the analyte to form a uniform suspension.

20. A method according to claim 14 wherein the structured surface comprises a region of a first material which is surrounded by a region of a second material so as to confine the mixture to the structured region.

21. A method according to claim 14 wherein the fluid comprises water and methanol.

22. A method according to claim 14 wherein the fluid comprises 10% water and 90% methanol.

23. A composition for use in performing a diagnostic assay of an analyte, wherein the composition comprises:
a fluid that wets a structured surface, the analyte being mixed into the fluid.

24. A composition according to claim 23 wherein the fluid is a solvent for the analyte.

25. A composition according to claim 23 wherein the analyte is uniformly suspended in the fluid.

26. A composition according to claim 23 wherein the fluid comprises a mixture of two or more fluids, at least one of which wets a structured surface.

27. A composition according to claim 26 wherein at least one fluid wets a structured surface and at least one fluid mixes with the analyte to form a solution.

28. A composition according to claim 26 wherein at least one fluid wets a structured surface and at least one fluid mixes with the analyte to form a uniform suspension.

29. A composition according to claim 23 wherein the fluid comprises water and methanol.

30. A composition according to claim 23 wherein the fluid comprises 10% water and 90% methanol.
